(19) United States
(12) Patent Application Publication
(10) Pub. No.: US 2005/0266096 A1
(43) Pub. Date: Dec. 1, 2005

(54) New synergetic compositions of vitamins, minerals and trace-elements to stimulate the elimination of intra-cellular lipid deposits

(76) Inventors: Ravi Shrivastava, Cebazat (FR); M. Hema Belani, Pune (IN)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(21) Appl. No.: 10/689,515

(22) Filed: Jun. 1, 2004

(51) Int. Cl. A61K 33/34; A61K 33/32; A61K 33/04

(52) U.S. Cl. 424/638; 424/641; 424/702; 514/251; 514/276; 514/356; 514/458

(57) ABSTRACT

Nearly 40% population of the developed countries in the world have the problem of excess weight. Up till now, no correlation was established between the deficiencies of certain specific vitamins , minerals and trace-elements with the problem of excess weight. None of the weight reduction strategies proposed till today took into consideration the reduced capacity of the cells to eliminate the intracellular lipid deposits which is the real cause of excess weight. After many years of research, we discovered that a synergetic composition of certain specific vitamins, minerals and trace-elements stimulate the intracellular lipid elimination capacity of cells thereby helping to reduce body weight in a very short period of time without any side-effects and without the rebound effect often observed at the end of the dieting period. The methods of preparation of such solid or liquid compositions for oral or topical application are known. These synergetic compositions can be used either alone or in association with products commonly used to reduce weight, with specific diets, with apparatuses for physical exercise or muscular electrostimulation which are commonly employed to increase energy loss.

The present invention concerns the use of a new synergetic compositions of vitamins, minerals and trace-elements to stimulate the elimination of intracellular lipid deposits.

Vitamins, minerals and trace-elements are indispensable micro-nutrients to regulate metabolic functions of the body. Vitamins are organic substances with variable and sometimes complex chemical structures, while minerals and trace-elements have a mineral origin.

Each of these micro-nutrients have a specific role (constitutive, catalytic, co-enzyme, pro-hormone) which can not be replaced by an another element to maintain optimal cellular functions.

The active doses are variable. For example the vitamins are required in small doses, the proportions for each of them varies between 1 to 25 000, if one takes extremes that are respectively the vitamin B12 for weakest and the C vitamin for strongest requirement. The same is true for the trace-elements.

The values selected by different experts taking into account the nutritional requirements, the purpose and the dietary habits of different group of persons concerned (children, teenagers, adults, aged people, sportsman, etc.) helped to establish nutritional needs (DUPIN H. ABRAHAM J. GIACHETTI I. Nutritional requirements for the French population, Tec & Doc Lavoisier, Paris, 1999) or to establish Recommended Daily Allowances (RDA).

In spite of the existence of these recommendations, a growing number of epidemiological studies conducted in the developed countries of the world demonstrate a more or less marked deficiency of micro nutrients the existence of defaulting more or less marked according to micro-nutrients and the segment of population considered.

According to results of three main investigations on the nutritional status of the French population, the percentage of the population that doesn't receive Recommended Daily Allowances is as follows (SOUCCAR T, CURTAY J. P, The new guide of vitamins, Seuil Editions, Paris, 1996, page 11):

| Vitamins | % Men not | % Women not | |
|---|---|---|---|
| Minerals & | receiving | receiving | |
| Trace-elements | RDA | RDA | Mean |
| Vitamin A | 11.7 to 60 | 8 to 50 | 32.43 |
| Vitamin B1 | 43 to 80 | 69 to 80 | 68.00 |
| Vitamin B2 | 27 to 60 | 24.6 to 60 | 42.90 |
| Vitamin B3 | 49.5 | 49.3 | 49.40 |
| Vitamin B6 | 67.5 to 80 | 90 to 92 | 82.38 |
| Vitamin B9 | 40 to 90 | 50 to 90 | 67.50 |
| Vitamin C | 25 to 60 | 15 to 60 | 40.00 |
| Vitamin D | 90 to 98 | 90 to 98.6 | 94.15 |
| Vitamin E | 40 to 100 | 75 to 100 | 78.75 |
| Magnesium | 60 | 80 | 70.00 |
| Calcium | 20 | 30.0 | 25.00 |
| Iron | 5 | 55 to 90 | 38.75 |
| Zinc | 80 | 90 | 85.00 |
| | | Total | 59.56 |

These studies demonstrate that on an average 6 to 7 people out of 10 show a more or less marked deficiency for a vitamin, a mineral or a trace-element but the exact consequences of these deficiencies are not scientifically established.

On the other hand, there is no food that contains all these vital elements in sufficient quantity and in adequate proportion.

The exact and correct role of vitamins, minerals and trace-elements are still not completely understood. The exact proportions of these different vital elements required to stimulate specific vital cellular functions are also not yet scientifically well established.

Till today, there is no scientific study establishing a correlation between the deficiency in vitamins, minerals or trace-elements and the problem of excess weight or hypercholesterolemia.

A considerable increase in the proportion of individuals having excess weight (nearly 40% of the population) have been observed in most of the developed countries of the world.

Overweight is the consequence of the accumulation of large quantities of fat in certain cells of the body called adipocytes. These cells can store big quantities of lipids inside their cytoplasm, known as intracellular lipids. In fact, this excessive accumulation is related to the reduced capacity of the cells to eliminate the waste products and the lipids.

Different strategies were proposed to reduce weight such as specific diets (hypocaloric, protein rich diet), taking plant extracts to “burn fat”, drugs to reduce hunger sensation (isomerides), hormonal treatments, medical assistance . . . etc. These treatments induce physiological disturbances which may worsen the deficiency of certain vital elements, give temporary results, disturb normal cellular physiology, cause unwanted or toxic effects or induce a rebound effect commonly called as “Yo-yo” phenomenon.

Paradoxically, none of these general strategies take into consideration the heart of the problem which resides in the abnormal metabolic functions of adipocytes leading to the accumulation of intracellular lipids and causing subsequent excess weight.

Currently there is no treatment which takes into consideration the reduced capacity of the cells leading to intracellular lipid deposits which is the real cause of excess weight.

The present invention concerns the oral or local use of synergetic compositions containing specific vitamins, minerals and trace-elements to specifically stimulate the cellular elimination functions to reduce intracellular lipid accumulation without inducing any toxic or side-effect.

All topically applied caffeine containing slimming products act on the subcutaneous fat reserves but do not affect the fat accumulated in the deeper tissues. They have only a superficial effect which do not help to reduce weight.

Different orally used dietary or pharmaceutical compositions contain natural or synthetic products or plant extracts which are known to act on the excess weight. These compositions associate, for example, different plant extracts having diuretic, draining effects or plants which swells in the stomach or in the digestive tract to decrease the sensation of hunger but these compositions do not have a specific effect on the intracellular lipid deposits. The oral use of pharmacological active principals (caffeine, ephedrine, alpha and beta agonists) in dietary or nutritional supplements is prohibited or legally restricted. The mode of action of these drugs is central (hunger inhibition) and they have many side-effects.

Finally, many formulations containing vitamins, minerals and trace-elements are sold in the market but these compositions are either non-specific, have a wide range of action or are directed to a specific age of population (children, teenagers, adults, ladies, elderly persons) but there is no specific synergetic composition of vitamins, minerals and trace-elements which specifically stimulate the intracellular lipid elimination capacity of the cells aimed to reduce weight without inducing any toxicity.

We discovered with astonishment that the excess weight can be reduced in a very short period of time using synergetic compositions of certain specific vitamins, minerals and trace-elements which allow to stimulate the elimination of intracellular lipid reserves without any side-effect.

A synergetic association of vitamins, minerals and trace-elements which strongly and specifically stimulates the removal of intracellular lipid deposits was never described.

An increase intracellular lipid elimination capacity of the cells reduces cellular fat reserves, the total body mass and the weight. The activity of these vitamins, minerals and trace-elements specific compositions can still be enhanced when they are associated with plant extracts known to act locally and orally on excess weight, or with a specific diet.

In vitro, the accumulation of intracellular lipid reserves was reduced by using synergetic composition of vitamins, minerals and trace-elements, at doses between 15 and 200 percent of Recommended Daily allowances (RDA), particularly 50% of RDA.

Best results were observed with specific associations containing 4 specific vitamins and 4 minerals or trace-elements: vitamin A, vitamin B2, vitamin B9, vitamin E, magnesium, zinc, copper and selenium (89% reduction in the intracellular lipid accumulation).

The results of a 30-day clinical trial on subjects having body mass 20% above normal limits have shown that the use of a synergetic composition containing at least two vitamins among the vitamin A, B2, B9, E, with minimum two minerals or trace-element among the magnesium, zinc, copper, selenium allowed to reduce the mean body weight by −2.47 kg. The use of this composition was associated with a marked increase in the index of well-being parameters. The results on the body weight loss are better than the use of hypocaloric diet alone. The efficiency of this synergetic composition of vitamins, minerals and trace-elements is slightly enhanced when it is used in association with a nutritional supplement containing plant extracts known to help weight loss or with dieting. Moreover, the weight of volunteers using synergetic composition of vitamins, minerals and trace-elements remained stable during three months post treatment observation period, which was not the case with subjects who took only hypocaloric diet. This results shows that the use of synergetic composition allows to stabilise weight finally to prevent effect commonly observed after dieting.

That is why the current invention concerns the use of synergetic compositions containing vitamins, minerals and trace-elements which helps to stimulate intracellular lipid elimination capacity to reduce excess weight either or not in association with plant compositions which help weight loss, with specific diet, with physical exercise or muscle electrostimulation apparatuses.

The vitamins, minerals and trace-elements used to prepared these compositions can be obtained from different sources to be associated in different galenic forms commonly employed and known by professionals.

In the present invention, the term “composition” includes solid and non-solid (liquids, semi-liquid, viscous) preparations.

Under preferential mode of preparation, the compositions are pharmaceutical or nutritional preparations for oral use.

Under preferential mode of preparation, such solid compositions can also be prepared as lyophilised or non-lyophilised powders, instant powders (soup), as extruded filaments (granules, pasta), as sachets, capsules or tablets.

Under preferential mode of manufacturing, the non-solid compositions can also be presented as drinks or concentrated drinks, syrup or ampoules.

Under preferential mode of preparation, the compositions are pharmaceutical or cosmetic preparations for topical application.

These compositions can also be prepared as instant or slow release topical pharmaceutical preparations (cream, gel, dermal patches).

The composition to stimulate intracellular lipid elimination capacity contains agents allowing the release of vitamins, minerals and trace-elements in the body.

For the preparation of these compositions, the ingredients employed to supply vitamins can be selected from synthetic or natural sources of vitamins or from an extract rich in vitamins.

For the preparation of these compositions, the ingredients employed to supply minerals and in trace-elements can be selected from synthetic or natural sources of minerals and trace-elements, from an extract rich in minerals or trace-elements, on the yeast enriched with these elements.

Such ingredients are commercially available from QUIMDIS company (France) or from COOPER company (French Pharmaceutical association, France).

The doses may vary according to the type of composition selected and can be administered daily for a period of minimum 14 days or preferably 30 days at doses supplying 15 to 200 percent and preferably 50% of Recommended Daily Allowances (RDA) of each of these elements.

The RDA represents the true quantity of the micro-nutriment available to the body and not simply the gross weight of the micro-nutriment used.

Under preferential conditions of use, this composition is used to accelerate the elimination of intracellular lipids, to detoxify the cell with the aim to reduce weight, to reduce obesity, stress, tiredness, to enhance physical form, sleep, age related diseases or sexual desire.

According to the problem treated, these compositions can be associated with a drug, with diet or hypocaloric food, a nutritional or cosmetic product to obtain synergetic results.

This is why the present invention also includes the association of synergetic compositions to stimulate intracellular lipid elimination capacity of the cells with a composition containing plants or ingredients known to reduce body weight.

Under preferential conditions of use of these compositions, they are associated with different active substances, particularly with plant extracts.

The term “plant extract” represent plants as well as parts of the plants, for example, dried and dehydrated plants, powdered plants, extracts of such plant or even certain parts of the plant obtained using at least one aqueous and/or organic solvent which are used in classical forms in liquid or solid, pharmacy or dietary preparations.

Under preferential conditions of use, the plants extracts or the ingredients known to act on the body weight are selected among the group of plant extracts containing thermogenetically active principles, choleretic substances, ingredients able to trap fat at intestinal level or the stimulators of the intestinal transit.

Therefore, according to this invention, the compositions can contain one more substances capable to stimulate thermogenesis, particularly caffeine or a plant extract rich in caffeine such as the guarana seeds (Paullinia cupana), green tea (Thea senensis) extract, cola (Cola nitida) extract, choleretic substances particularly cynarin or artichoke extract (Cynara scolymus), certain spice plants such as the ginger (Zingiber officinalis), or an extract of ananas (Ananas comosus) rich in bromelaine, extract of papaya (Carica papaya) rich in papaine, vegetable fibres specially fruit fibres (lemon, grapefruit, apple), cereals, pectin, fructo-oligosaccharrides preferably inulin, chitosan, or clay preferably green clay of different origins.

Such active plant ingredients are sold by BIOSPHERE (France) or GREENTECH (France) or COOPER companies (French Pharmaceutical Co-operation, France).

These associations can be presented in the form of lyophilised or non-lyophilised powders, as small pieces (granules, pasta, extrudes), as sachets, capsules or tablets.

These associations can also be presented as pharmaceutical or cosmetic preparations for topical application such as creams, gels, or dermal patches.

This present invention also includes associating compositions capable to stimulate elimination of intracellular lipids with a diet, with an apparatus for physical exercise, or muscle electrostimulator.

Under preferential conditions of use the dietary regimens can be selected among the group of hypocaloric diets, protein rich diets or the diets enhancing intestinal transit.

Under preferential conditions of use, the apparatus for physical exercise or muscular electrostimulation can be an apparatuses or machines forcing muscular functions to enhance energy loss.

The following examples show some of the synergetic compositions according to this invention. These examples of compositions to stimulate the elimination of intracellular lipid deposits were developed on the basis of results of the pharmacological studies. Some of the selected compositions were then studied clinically.

### EXAMPLE 1

The capsules, tablets and sachets of vitamins, minerals & trace-elements were prepared according to the following formula (in mg/capsule, per tablet or per sachet).

| | |
|---|---|
| Vitamin A (Beta carotene) | 24.0000 |
| Vitamin B2 (riboflavin) | 0.8000 |
| Vitamin B9 (folic acid) | 0.1000 |
| Vitamin E (powder containing 50% vitamin E) | 10.0000 |
| Magnesium (magnesium oxide) | 248.7000 |
| Zinc (zinc oxide) | 9.3350 |
| Copper (copper oxide) | 1.2520 |
| Selenium (sodium selenite) | 0.0822 |
| Excipient for capsules qsp | 330 mgs |
| Excipient for tablets qsp | 400 mgs |
| Excipient for sachets qsp | 1400 mgs |

Posology: 1 capsule or 1 tablet every morning with a glass of water or 1 sachet to be mixed to a yoghurt or any other food.

### EXAMPLE 2

We have prepared capsules of vitamins, minerals and trace-elements associated with plant extracts and corresponding to the following formula:

| | milligrams/capsule |
|---|---|
| Vitamin A (beta carotene) | 24.0000 |
| Vitamin B2 (riboflavin) | 0.8000 |
| Vitamin B9 (folic acid) | 0.1000 |
| Vitamin E (powder containing 50% of vitamin E) | 10.0000 |
| Magnesium (magnesium oxide) | 248.7000 |
| Zinc (zinc oxide) | 9.3350 |
| Copper (copper oxide) | 1.2520 |
| Selenium (sodium selenite) | 0.0822 |
| Guarana extract containing 25% natural caffeine: | 25.0000 |
| Artichoke extract containing 1% cynarine: | 10.0000 |
| Excipient | qsp 365 mgs |

Posology: 1 capsule every morning with a glass of water.

### EXAMPLE 3

We have prepared capsules of vitamins, minerals and trace-elements associated with plant extracts and corresponding to the following formula:

| | milligrams/tablet |
|---|---|
| Vitamin A (beta carotene) | 24.0000 |
| Vitamin B2 (riboflavin) | 0.8000 |
| Vitamin B9 (folic acid) | 0.1000 |
| Vitamin E (powder containing 50% vitamin E) | 10.0000 |
| Magnesium (magnesium oxide) | 248.7000 |
| Zinc (zinc oxide) | 9.3350 |
| Copper (copper oxide) | 1.2520 |
| Selenium (sodium selenite) | 0.0822 |
| Cola walnut extract containing 25% caffeine: | 20.0000 |
| Pineapple extract | 10.0000 |
| Grapefruit fibres | 10.0000 |
| Excipient | qsp 400 mgs |

Posology: 2 tablets every morning with a glass of water.

### EXAMPLE 4

We have prepared vitamins, minerals and trace-elements sachets associated with plants or with ingredients known to act on excess weight corresponding to the following formula:

| | milligrams/sachet |
|---|---|
| Vitamin A (beta carotene) | 24.0000 |
| Vitamin B2 (riboflavin) | 0.8000 |
| Vitamin B9 (folic acid) | 0.1000 |
| Vitamin E (powder containing 50% vitamin E) | 10.0000 |
| Magnesium (magnesium oxide) | 248.7000 |
| Zinc (zinc oxide) | 9.3350 |
| Copper (copper oxide) | 1.2520 |
| Selenium (sodium selenite) | 0.0822 |
| Chicory inulin | 100.2160 |
| Guarana extract containing 25% natural caffeine | 25.000 |
| Artichoke extract containing 1% cynarine | 10.0000 |
| Green tea extract containing 3% natural caffeine | 10.0000 |
| Green clay | 50.0000 |
| Powdered pineapple juice | 10.0000 |
| Tamarind | 7.0000 |
| Ginger | 7.0000 |
| Karaya gum | 6.0000 |
| Excipient | qsp 1400 mgs |

Posology: Take 1 sachet per day with a glass of water or mixed with a yoghurt, preferably in the morning.

### EXAMPLE 5

We have prepared capsules of vitamins, minerals and trace-elements corresponding to the following formula:

| | milligrams/capsule |
|---|---|
| Vitamin A (beta carotene) | 8.0000 |
| Vitamin B1 (thiamin hydrochloride) | 0.2350 |
| Vitamin B2 (riboflavin) | 0.2670 |
| Vitamin B3 (nicotinamid) | 3.0000 |
| Vitamin B5 (pantothenic acid) | 1.0000 |
| Vitamin B6 (pyridoxine hydrochloride) | 0.3350 |
| Vitamin B8 (biotin) | 0.0250 |
| Vitamin B9 (folic acid) | 0.0350 |
| Vitamin B12 (cyanocobalamine) | 0.000165 |
| Vitamin C (ascorbic acid) | 20.0000 |
| Vitamin E (powder containing | 3.3350 |
| 50% vitamin E) | |
| Magnesium (magnesium oxide) | 83.0000 |
| Calcium (calcium carbonate) | 333.2000 |
| Iron (iron gluconate) | 18.6400 |
| Iodine (potassium iodide) | 0.0327 |
| Potassium (potassium chloride) | 24.4900 |
| Zinc (zinc oxide) | 3.1120 |
| Copper (copper oxide) | 0.4170 |
| Selenium (sodium selenite) | 0.0274 |
| Excipient | qsp 540 mgs |

Posology: 3 capsules per day with a glass of water, preferably in the morning.

### EXAMPLE 6

We have prepared pasta containing an association of vitamins, minerals and trace-elements corresponding to the following formula:

| | Milligrams for |
|---|---|
| | 100 grams of pasta |
| Vitamin A (beta carotene) | 24.000 |
| Vitamin B2 (riboflavin) | 0.8000 |
| Vitamin B9 (folic acid) | 0.1000 |
| E vitamin (powder containing 50% of vitamin E) | 10.0000 |
| Magnesium (magnesium oxide) | 248.7000 |
| Zinc (zinc oxide) | 9.3350 |
| Copper (copper oxide) | 1.2520 |
| Selenium (sodium selenite) | 0.0822 |
| Fresh egg powder | 32 000.0000 |
| Crushed wheat | qsp 100 grams |

Posology: water cooked 100 to 200 grams of pasta per day.

### EXAMPLE 7

Association of the synergetic composition given in the example 1 with 10 grams of dehydrated soup containing coli flower, vegetables and milk proteins, to be taken ad-libitum. This preparation is directed to a hypocaloric diet poor in carbohydrates & fat, rich in proteins & dietary fibres, supplying about 32 kilo-calory for 10 g of dehydrated soup.

### EXAMPLE 8

Association of the synergetic composition given in the example 1 with 100 grams of protein enriched pasta to be taken at mid-day and evening with green vegetables, ad-libitum.

### EXAMPLE 9

Association of a synergetic composition of the example 1 with sachets of 1.4 grams each, containing plants known to act on excess body weight corresponding to the following formula:

| | Percentage weight |
|---|---|
| Chicory inulin | 82.555 |
| Pre-cooked soy | 2.500 |
| Wheat germ | 1.675 |
| Artichoke extract containing 3% cynarin | 0.950 |
| Guarana extract containing 10% caffeine | 0.570 |
| Green tea extract containing 3% caffeine | 0.570 |
| Pre-cooked oat | 0.250 |
| Apple fibres | 0.100 |
| Seaweed | 0.100 |
| Yeasts | 0.025 |
| Flavour | 0.095 |
| Excipient | qsp 100% |

Posology: Take 1 sachet per day with a glass of water or mixed with a yoghurt preferably the morning.

### EXAMPLE 10

Combination of synergetic composition as given in the example 1 with a slimming gel corresponding to the following formula:

| | Percentage weight |
|---|---|
| Caffeine | 2.0 |
| Centella asiatica extract | 2.0 |
| Mimosa tenuiflora extract | 1.5 |
| Azadirachta indica extract | 0.6 |
| Garcinia cambogia extract | 5.5 |
| Excipients | qsp 100% |

Posology: Apply a small amount of product every morning and evening, enhance skin penetration by slight massage.

### EXAMPLE 11

We prepared a dermal patch system of 10 cm allowing to diffuse every day sufficient quantities of vitamins, minerals and trace-elements indicated below and corresponding to the following formula:

| | milligrams/day |
|---|---|
| Vitamin A (beta carotene) | 24.000 |
| Vitamin B2 (riboflavin) | 0.8000 |
| Vitamin B9 (folic acid) | 0.1000 |
| Vitamin E (powder containing 50% vitamin E) | 10.0000 |
| Magnesium (magnesium oxide) | 248.7000 |
| Zinc (zinc oxide) | 9.3350 |
| Copper (copper oxide) | 1.2520 |
| Selenium (sodium selenite) | 0.0822 |
| Transdermal adhesive system and excipients | qsp 500 mgs |

### EXAMPLE 12

Combination of the composition of the example 11 with a transdermal system of 10 cm containing plant extracts known to reduce body weight corresponding to the following formula:

| | milligrams/day |
|---|---|
| Caffeine | 10.0 |
| Guarana extract | 20.0 |
| Fucus extract | 10.0 |
| Dermal adhesive patch and excipient | qsp 500 mgs |

The current invention also includes the preparation of compositions given above or the synergetic association of the ingredients given in above compositions with acceptable excipients or solvents, particularly pharmaceutical, cosmetic or dietary excipients, according to usual methods of associating such compositions.

### Tolerance Studies

The acute toxicity studies in rats, performed according to the OECD 401 guidelines under good laboratory practice norms show that the preparations given in the examples are not toxic by oral route up to a dose of 5 g per kg body weight.

The primary skin irritation and the ocular irritation studies in rabbits conducted according to the official method of skin irritation test (European regulations of 11^{th} May 1993) and according to the official method of ocular irritation test (European regulations of 9^{th} June 1992) under quality assurance show that the examples cited above are non-irritant when applied topically.

### Pharmacological Studies: Evaluation of the Association of Ingredients Capable to Stimulate Cellular Elimination Capacity to Reduce Intracellular Lipid Accumulation

Cell Cultures

To evaluate the effects of vitamins, minerals & trace-elements on the cellular capacity to eliminate intracellular lipid deposits, the cultures of vascular smooth muscle cells were prepared on plastic cover slips in vitro as described by Shrivastava at al (Meth Expt Find,1994).

A 50-60% cell monolayer was obtained within 4 days of culture. The hyperlipidemic rabbit serum was added to the culture medium, cells were incubated at 37° C.-5% CO₂ for 48 h and the presence of intracellular cytoplasmic lipid deposit was quantified (considered 100% lipid deposits) . The culture medium was then replaced by a predefined blank medium containing no vitamins, minerals & trace-elements containing 1% fetal calf serum. Desired concentrations of vitamins, minerals and trace-elements were prepared in the predefined control culture medium by dissolving the test substances in the medium and by replacing the culture medium by the test product containing medium.

The in vitro cytoxicity of each vitamin, mineral and trace-element was determined. None of the concentrations used was cytotoxic. The test product concentration tested was 1/100^{th} of the RDA of the basic test product per ml in the culture medium.

The following test products and their combinations were tested: Vitamin A (beta carotene), vitamin B1 (thiamin hydrochloride), vitamin B2 (riboflavin), vitamin B3 or PP (nicotinamid), vitamin B5 (pantothenic acid), vitamin B6 (pyridoxine hydrochloride), vitamin B8 or H (Biotin), vitamin B9 (folic acid), vitamin B12 (cynacobalamin), vitamin C (ascorbic acid), vitamin E (tocopherol acetate), iron (iron gluconate), iodine (potassium iodide), potassium (potassium chloride), zinc (zinc oxide), copper (copper oxide), magnesium (magnesium oxide), calcium (calcium gluconate) ,and selenium (selenium selenite). Only the elements showing some activity during the preliminary screening were selected for further testing.

The % of intracellular lipid deposits was evaluated 3-days after test product exposure according to the method described by Shrivastava et al. (Meth. Find. Exp. Clin. Pharmacol. 15, 345-350, 1993). The results were expressed a mean percentage decrease in intracellular lipid deposits compared to the untreated control cultures considered as containing 100% lipid deposits.

The following results were obtained with individual substance (table 1), with the association of two substances (table 2) and with the synergetic association of different micro-nutrients (table 3).

TABLE 1
Percentage decrease in intracellular lipid deposits
(n = 24) with individual element.

| | Concentration tested | Percent decrease in |
|---|---|---|
| Element | in μg/ml (0.016% RDA) | intracellular lipids |
| Vitamin A | 8.0 | 12.6 ± 2.3 |
| Vitamin B1 | 0.23 | 7.7 ± 1.4 |
| Vitamin B2 | 0.26 | 14.5 ± 3.9 |
| Vitamin B3 | 3.0 | 2.5 ± 1.0 |
| Vitamin B5 | 1.0 | 4.5 ± 2.1 |
| Vitamin B6 | 0.335 | 9.9 ± 2.9 |
| Vitamin B8 | 0.025 | 1.8 ± 0.7 |
| Vitamin B9 | 0.035 | 17.5 ± 4.6 |
| Vitamin B12 | 0.0001 | 8.2 ± 2.5 |
| Vitamin C | 20.0 | 11.2 ± 4.1 |
| Vitamin E | 3.33 | 21.4 ± 3.9 |
| Magnesium | 66.67 | 22.0 ± 5.2 |
| Calcium | 178.0 | 0.0 |
| Iron | 18.64 | 6.3 ± 2.7 |
| Iodine | 0.03 | 0.0 |
| Potassium | 24.49 | 0.0 |
| Zinc | 3.11 | 16.3 ± 4.4 |
| Copper | 0.42 | 24.3 ± 6.2 |
| Selenium | 0.013 | 24.4 ± 5.3 |

These results show that these products tested alone do not have any effect on the reduction of intracellular lipid accumulation except minor effect for the vitamin A, vitamin B2, vitamin B9, vitamin E, magnesium, zinc, copper and selenium.

TABLE 2
% decrease in intracellular lipid deposits with
association of two ingredients (n=24)

| | % decrease in |
|---|---|
| Association | intracellular lipids |
| Vitamins A + B2 | 20.2 ± 4.6 |
| Vitamins A + B9 | 26.3 ± 5.5 |
| Vitamins A + E | 29.2 ± 6.7 |
| Vitamin A + Magnesium | 26.1 ± 4.4 |
| Vitamin A + Zinc | 18.3 ± 4.4 |
| Vitamin A + Copper | 21.3 ± 9.1 |
| Vitamin A + Selenium | 17.2 ± 6.1 |
| Vitamins B2 + B9 | 18.3 ± 2.7 |
| Vitamins B2 + E | 20.0 ± 4.1 |
| Vitamin B2 + Magnesium | 30.2 ± 8.1 |
| Vitamin B2 + Zinc | 21.1 ± 6.6 |
| Vitamin B2 + Copper | 23.3 ± 7.3 |
| Vitamins B9 + E | 24.4 ± 6.6 |
| Vitamin B9 + Magnesium | 18.1 ± 8.2 |
| Vitamin B9 + Zinc | 18.4 ± 3.2 |
| Vitamin B9 + Copper | 22.6 ± 4.9 |
| Vitamin E + Magnesium | 25.9 ± 4.9 |
| Vitamin E + Zinc | 26.6 ± 7.6 |
| Vitamin E + Copper | 17.7 ± 2.7 |
| Zinc + Copper | 16.6 ± 4.9 |
| Zinc + Magnesium | 21.9 ± 7.2 |
| Copper + Magnesium | 24.6 ± 6.2 |
| Vitamin B2 + Selenium | 24.6 ± 2.7 |
| Vitamin E + Selenium | 22.2 ± 7.8 |
| Magnesium + Selenium | 23.7 ± 8.2 |

These results show that the association of two vitamins, mineral or trace-elements has no additional effect on the elimination of intracellular lipid deposits. This effect is not additive. The activity of the association of magnesium with vitamin B2 is slightly better than the two elements taken alone.

TABLE 3
% decrease in the intracellular lipid deposits with the association
of different micro-nutrients (n = 24) eventually combined
with plant extracts (vitamin A = A, vitamin B2 = B2
and so on for other vitamins; the minerals and trace-elements
are represented by international symbols).

| | % decrease in |
|---|---|
| | intracellular |
| Associations | lipids |
| A + B2 + B9 + E | 26.4 ± 6.9 |
| A + B2 + Mg + Zn | 70.0 ± 9.3 |
| A + B5 + B6 + B8 | 22.4 ± 7.6 |
| A + Fe + I + K | 15.3 ± 4.3 |
| B9 + Fe + Mg + Zn | 31.2 ± 8.4 |
| A + B2 + Cu + Se | 68.4 ± 8.8 |
| A + B2 + Mg + Se | 81.2 ± 11.4 |
| B5 + B6 + Cu + Se | cytotoxic |
| B2 + B9 + Mg + Zn | 68.8 ± 11.4 |
| B2 + B9 + Zn + Se | 77.3 ± 9.9 |
| B2 + B9 + Mg + Se | 76.3 ± 13.2 |
| B2 + B9 + Cu + Se | 80.4 ± 8.2 |
| B2 + Mg + Cu + Se | 34.9 ± 6.7 |
| B5 + B6 + B8 + Fe + I | 24.6 ± 7.2 |
| A + B2 + Mg + Cu | 76.3 ± 14.6 |
| B2 + B9 + E + Zn | 42.6 ± 14.6 |
| D + E + Zn + Si | 52.0 ± 9.0 |
| E + Ca + Fe + Cr | 36.4 ± 8.8 |
| A + B2 + B9 + E + Mg + Zn + Cu + Se | 89.2 ± 12.6 |
| A + B2 + B9 + Mg + Zn + Cu + Se | 72.1 ± 11.6 |
| B2 + B9 + E + Mg + Zn + Cu + Se | 81.6 ± 12.0 |
| A + E + Mg + Zn + Cu + Se | 71.6 ± 9.9 |
| A + E + Zn + Cu + Se | 61.4 ± 13.0 |
| E + Mg + Zn + Cu + Se | 40.1 ± 8.8 |
| A + B2 + Mg + Cu + caffeine (0.01%) | 84.4 ± 14.6 |
| A + B2 + Mg + Cu + Soy phytoestrogens (10%) | 83.4 ± 17.3 |

These results demonstrate that to remove intracellular lipid accumulation, it is indispensable to associate minimum two vitamins among the vitamins A, B2, B9, E and minimum two minerals among magnesium, zinc, copper and selenium. Best results were obtained by combining the eight elements.

The activity reduces when minimum two elements from each side are not associated.

### Clinical Trial: Confirmation of Results in Humans Being

Capsules, tablets or sachets containing 50% RDA of vitamin A, B2, B9, E, magnesium, zinc, copper and selenium, were prepared (1 capsule, or 1 tablet or 1 sachet were used per day according to the type of the form used).

Adult volunteers, either male (about 35%) or female (about 65%) having an IMC above 20% than normal were selected and were given different regimens to evaluate the effect on body weight over a period of 30-days. The weight of volunteers was also determined 3-months after the date of the start of this study.

Decrease in weight was considered to be relate to the increase in the cellular metabolic functions leading to the elimination of waste products from the body and subsequent weight loss. The effect on well being parameters was evaluated on a 0-4 scale score. Five identical groups were constituted and the parameters were measured by the Vitro-Bio research institute recognised by the French Ministry of Research.

Group 1: Only vitamins, minerals and trace-element nutritional supplement as given in the example 1.

Group 2: Association of vitamins, minerals and trace-element nutritional supplement with a plant based nutritional supplement as given in the example N°9.

Group 3: Association of vitamins, minerals and trace-element nutritional supplement with a regimen containing ad-libitum fruits, green vegetables and 100 g protein rich pasta, in the morning and evening, as given in the example 8.

Group 4: Regimen based on a lyophilised vegetable soup to be prepared instantly with hot water as a soup.

Group 5: combination of a food supplement containing vitamins, minerals and trace-elements associated with a regimen of lyophilised vegetable soup to be mixed with boiling water for instant preparation.

The following results were obtained (mean values):

| | | | Mean weight | | | |
|---|---|---|---|---|---|---|
| | Number of | | loss within | | | Index of |
| | volunteers | | 30-days | | | well being |
| | Men | Women | Men | Women | Average | (1-4 scale) |
| Group 1 | 8 | 30 | −2.22 | −2.61 | −2.47 | 3.21 |
| Group 2 | 9 | 18 | −3.67 | −3.82 | −3.76 | 3.42 |
| Group 3 | 4 | 12 | −2.10 | −2.93 | −2.62 | 3.11 |
| Group 4 | 7 | 14 | −1.15 | −1.30 | −1.22 | 1.8 |
| Group 5 | 12 | 18 | −2.20 | −2.75 | −2.56 | 2.89 |

These results show that the association of the 50% RDA of vitamin A, B2, B9, E with Magnesium, zinc, copper and selenium reduce weight both in men and women (mean −2.47 Kg in 30 days) by accelerating the elimination of intracellular lipids. The results are better when this synergetic composition is combined with a plant based nutritional supplement. These results can also be enhanced by combination of different regimens but the additional weight loss observed is not always significant showing that the major part of the weight loss is due to synergetic composition of vitamins (A, B2, B9, E), minerals and trace-elements (magnesium, zinc, copper and selenium).

The volunteers of the group 4 taking only a diet based on vegetable soup have shown body weight loss which is much less compared to the groups taking synergetic composition of vitamins, minerals and trace-elements.

On the other side, it has been observed that the weight of the volunteers taking synergetic composition of vitamin, mineral and trace-element association remained stable at the end of the 3-month observation period which is not the case in the groups taking only hypocaloric diet. These results show that the use of this synergetic composition helps to stabilise weight and stops the rebound effect commonly seen at the end of the dieting period.

In volunteers taking synergetic composition of vitamins, minerals and trace-elements, the index of well being parameters was significantly increased with remarkable improvement in parameters of stress, sleep, physical form, and tiredness; showing that the synergetic association of vitamin A, vitamin B2, vitamin B9, vitamin E, magnesium, zinc, copper and selenium exert a favourable effect on health in general.

1. A composition containing at least two vitamins of vitamin A, vitamin B2, vitamin B9 or vitamin E along with at least two minerals or trace-elements of magnesium, zinc, copper and selenium in an amount effective for reduction of intracellular lipids and toxic wastes which have accumulated in a human body.

2. The composition according to claim 1 and further comprising a pharmaceutically acceptable excipient.

3. The composition of claim 1 and further comprising thermogenetic stimulating ingredients that have a cholagogue or choleretic effect or which are known to reduce body weight for treatment of obesity.

4. A method for reducing intracellular lipids and toxic wastes that have accumulated in the human body, the method comprising orally administering a composition according to claim 1.

5. A method for reducing intracellular lipids and toxic wastes that have accumulated in the human body, the method comprising orally administering a composition according to claim 2.

6. A method for reducing intracellular lipids and toxic wastes that have accumulated in the human body, the method comprising orally administering a composition according to claim 3.

7. The method of claim 4 wherein the oral administration is in combination with a hypocaloric diet, a protein rich diet, or a diet effective for stimulating intestinal transit.

8. The method of claim 5 wherein the oral administration is in combination with a hypocaloric diet, a protein rich diet, or a diet effective for stimulating intestinal transit.

9. The method of claim 6 wherein the oral administration is in combination with a hypocaloric diet, a protein rich diet, or a diet effective for stimulating intestinal transit.

10. The method claim 4 and further comprising physical exercise or muscular electro-stimulation with the oral administration.

11. The method claim 5 and further comprising physical exercise or muscular electro-stimulation with the oral administration.

12. The method claim 6 and further comprising physical exercise or muscular electro-stimulation with the oral administration.

13. The composition of claim 1 in the form of a pharmaceutical, cosmetic, dietary, or nutritional product.

14. The composition of claim 2 in the form of a pharmaceutical, cosmetic, dietary, or nutritional product.

15. The composition of claim 3 in the form of a pharmaceutical, cosmetic, dietary, or nutritional product.
